(19) United States
(12) Patent Application Publication
(10) Pub. No.: US 2026/0294436 A1
(43) Pub. Date: Oct. 1, 2026

(54) LEFT ATRIAL APPENDAGE OCCLUSION

(71) Applicants: Medtronic, Inc., Minneapolis, MN (US); Medtronic CryoCath LP, Toronto (CA)

(72) Inventors: Zhongping Yang, Woodbury, MN (US); Nicolas Coulombe, Anjou (CA)

(21) Appl. No.: 19/479,275

(22) PCT Filed: Apr. 24, 2024
(86) PCT No.: PCT/IB2024/053992

§ 371 Date: Oct. 27, 2025

Related U.S. Application Data
(60) Provisional application No. 63/462,546, filed on Apr. 28, 2023.

(51) Int. Cl. A61B 17/12; A61B 17/00

(52) U.S. Cl. CPC A61B 17/12122; A61B 17/00491; A61B 17/12136; A61B 2017/12054; A61B 2217/005

(57) ABSTRACT

A medical system, device and method are described for occluding a left atrial appendage. An example medical device, such as an occlusion device, may include a first catheter with an elongate tubular body and a central lumen that extends from a proximal end to a distal end arranged to be positioned at an opening of the left atrial appendage. A deployable balloon may be arranged at the distal end of the catheter and is operable to seal against an opening of the left atrial appendage. An aspiration source attached to the central lumen of the catheter is operable to at least partially evacuate and collapse the left atrial appendage. A steerable delivery catheter may be arranged to deliver adhesive within the partially collapsed left atrial appendage to thereby occlude the left atrial appendage.

This application is an international application claiming priority to U.S. provisional patent application No. 63/462,546, filed 28 Apr. 2023, the entire contents of which is incorporated herein by reference.

### FIELD OF THE INVENTION

The present invention relates to a method of occluding a left atrial appendage and an occlusion device for occluding a left atrial appendage.

### BACKGROUND OF THE INVENTION

The human heart is a four chambered, muscular organ that provides blood circulation through the body during a cardiac cycle. The four main chambers include the right atrium RA and right ventricle RV which supplies the pulmonary circulation, and the left atrium LA and left ventricle LV which supplies oxygenated blood received from the lungs into systemic circulation. To ensure that blood flows in one direction through the heart, atrioventricular valves (tricuspid valves TV and mitral valves MV) are present between the junctions of the atria and the ventricles, and semi-lunar valves (pulmonary valve and aortic valve) govern the exits of the ventricles leading to the lungs and the rest of the body.

The heart also includes a left atrial appendage LAA, which is a small, ear-shaped sac in the muscle wall of the left atrium LA. In normal hearts, when the heart contracts, the blood in the left atrium LA and the left atrial appendage LAA is squeezed out of the left atrium LA and into the left ventricle LV. The LAA has minimal influence on cardiac output and is generally considered to be non-functional structure in the heart.

In atrial fibrillation, an irregular heartbeat causes blood flow to slow enabling clots to form. Because the left atrial appendage LAA is a small sac or pouch, blood may collect there and form clots. The clots often cause thromboembolic complications and risk to the patient. This risk increases if the thrombus or fragments of the thrombus dislodge. If all or a portion of the thrombus flows downstream, it is highly likely that the free material will become trapped in smaller and more tortuous anatomy. This increases a patient's risk for cerebral stroke or peripheral embolism. Thus, in some cases, it may be desirable to exclude or occlude the left atrial appendage LAA such that clots do not form in the left atrial appendage LAA, and if they do, they cannot escape the left atrial appendage LAA. While medications can be taken to control blood clotting and decrease the chance of stroke, a proportion of patients cannot take such medications or follow the strict regimen necessary for continued protection.

To reduce the above-mentioned risks, it is desirable to close the left-atrial appendage to reduce the occurrence of thrombus formation and the risk of thromboembolism. The present disclosure relates to improvements in catheter-based occlusion systems for occluding or excluding the left atrial appendage.

### SUMMARY OF THE INVENTION

A first aspect of the invention provides a method of occluding a left atrial appendage, the method comprising: providing a catheter with an elongate tubular body and a central lumen that extends from a proximal end to a distal end of the catheter, the catheter comprising a deployable balloon arranged at the distal end of the catheter; positioning the catheter adjacent to an opening of the left atrial appendage; advancing the distal end of the catheter through the opening and into the left atrial appendage; inflating the balloon to contact an area surrounding the opening of the left atrial appendage to create a seal between the balloon and the left atrial appendage; evacuating the left atrial appendage to at least partially collapse the left atrial appendage; and inserting adhesive into the at least partially collapsed left atrial appendage to secure the left atrial appendage in the at least partially collapsed state.

Optionally, the left atrial appendage has a pressure after the balloon contacts an area surrounding the opening of the left atrial appendage, and wherein evacuating the left atrial appendage comprises creating a reduced pressure in the left atrial appendage to reduce a volume of the left atrial appendage.

Optionally, evacuating the left atrial appendage comprises aspirating through at least one aperture at the distal end of the catheter to create the reduced pressure in the left atrial appendage.

Optionally, the catheter is a cryoballoon catheter and fully inflating the balloon comprises at least partially filling the balloon with cryo fluid or refrigerant to contact the area surrounding the opening of the left atrial appendage.

Optionally, the cryoballoon is collapsed when the catheter is positioned adjacent to the opening of the left atrial appendage.

Optionally, the catheter further comprises an electrical sensor, and the method further comprises deploying the electrical sensor into the left atrial appendage and obtaining an electrical activity reading before and after the cryoballoon cryoadheres to the area surrounding the opening of the left atrial appendage.

Optionally, the catheter further comprises a pressure sensor at the distal end of the catheter to measure the pressure in the left atrial appendage and the method further comprises: observing a pressure reading at the distal end of the catheter and outputting an alarm if the observed pressure crosses a predetermined threshold.

Optionally, the method comprises incorporating radiopaque markers into the adhesive.

Optionally, the method further comprises curing the adhesive before collapsing the balloon to remove the seal between the balloon and the left atrial appendage.

Optionally, the adhesive is a visible light or ultraviolet cured (UV) adhesive, wherein the method further comprises using a light source to cure the adhesive after the adhesive is inserted into the at least partially collapsed left atrial appendage.

Optionally, the catheter further comprises the light source or wherein the method further comprises providing a further catheter comprising the light source.

Optionally, the catheter further comprises a securing mechanism arranged to engage with an area surrounding the opening of the left atrial appendage, the method further comprising deploying the securing mechanism to engage with the area surrounding the opening of the left atrial appendage to secure the left atrial appendage in the at least partially collapsed state.

Optionally, a portion of the distal end of the catheter is detachable and forms the securing mechanism, the method further comprising detaching the portion of the distal end of the catheter and engaging the detached portion with the area surrounding the opening of the left atrial appendage to secure the left atrial appendage in the at least partially collapsed state.

Optionally, the method further comprises: providing a steerable catheter comprising a securing mechanism arranged to engage with an area surrounding the opening of the left atrial appendage; and inserting a distal end of the steerable catheter towards the opening of the left atrial appendage before deploying the securing mechanism to engage with the area surrounding the opening of the left atrial appendage to secure the left atrial appendage in the at least partially collapsed state.

A further aspect of the invention provides an occlusion device for occluding a left atrial appendage, the device comprising: a first catheter with an elongate tubular body and a central lumen that extends from a proximal end to a distal end of the catheter, the distal end arranged to be positioned at an opening of the left atrial appendage; a deployable balloon arranged at the distal end of the catheter; wherein the deployable balloon is operable to seal against an opening of the left atrial appendage; a second catheter arranged to deliver adhesive at the distal end of the second catheter; and an aspiration source attached to the central lumen of the first catheter, wherein the aspiration source is operable to at least partially evacuate the left atrial appendage to at least partially collapse the left atrial appendage.

Optionally, the second catheter is housed in the central lumen of the first catheter.

Optionally, the deployable balloon is a cryoballoon.

Optionally, at least a portion of an outer surface of the cryoballoon is formed of a different material to the remaining outer surface of the cryoballoon.

Optionally, the first catheter further comprises at least one aperture at the distal end of the first catheter, wherein the at least one aperture extends perpendicularly from the central lumen so that the aspiration source is operable to at least partially evacuate the left atrial appendage through the at least one aperture.

Optionally, the first catheter further comprises a plurality of apertures along a longitudinal length of the first catheter, wherein the plurality of apertures extend perpendicularly from the central lumen, so that the aspiration source is operable to at least partially evacuate the left atrial appendage through the plurality of apertures.

Optionally, the first catheter comprises at least two apertures in a radial arrangement around a circumference of the tubular body at the distal end of the first catheter so that the aspiration source is operable to at least partially evacuate the left atrial appendage through the at least two apertures.

Optionally, the occlusion device further comprises a pressure sensor at a distal end of the first catheter, wherein the pressure sensor is arranged to measure pressure in the left atrial appendage, and optionally, the pressure sensor is arranged to send a signal to a control system if the pressure crosses a predetermined threshold.

Optionally, the occlusion device further comprises a light source that is arranged to cure the adhesive. optionally, the adhesive is bioglue.

Optionally, the occlusion device further comprises a deployable electrical sensor that is arranged to extend into a left atrial appendage to measure electrical activity in the left atrial appendage.

Optionally, the occlusion device further comprises a securing mechanism, wherein the securing mechanism is arranged to engage with an area surrounding the opening of the left atrial appendage to secure the left atrial appendage in the at least partially collapsed state.

Optionally, the securing mechanism forms part of the elongate tubular body of the first catheter, and wherein the securing mechanism is detachable from the first catheter.

Optionally, the occlusion device further comprises a steerable catheter comprising a securing mechanism arranged to engage with an area surrounding the opening of the left atrial appendage; and preferably, wherein the securing mechanism is detachable from the steerable catheter.

Optionally, the securing mechanism comprises a plurality of tines and/or barbs, wherein the tines and/or barbs are arranged to mechanically engage with an area surrounding the opening of the left atrial appendage to secure the left atrial appendage in the at least partially collapsed state.

### BRIEF DESCRIPTION OF THE DRAWINGS

Embodiments of the invention will now be described with reference to the accompanying drawings, in which:

FIG. 1 is a schematic sectional illustration of a heart;

FIG. 2 illustrates a diagrammatic view of a patient undergoing a cardiovascular procedure to occlude a left atrial appendage;

FIG. 3 illustrates an exemplary left atrial appendage;

FIG. 4 illustrates an exemplary catheter;

FIG. 5 illustrates an exemplary method of occluding a left atrial appendage;

FIG. 6 illustrates a catheter adjacent to a left atrial appendage;

FIG. 7A-7D are schematic views of a part of an occlusion device according to an embodiment;

FIG. 8 illustrates a distal end of a catheter inserting into the left atrial appendage;

FIG. 9 illustrates a catheter with an inflated balloon in a left atrial appendage;

FIG. 10 illustrates a partially evacuated and partially collapsed left atrial appendage;

FIG. 11 illustrates a partially collapsed left atrial appendage with adhesive;

FIG. 12 illustrates the distal end of a catheter with a delivery catheter;

FIGS. 13a-13c illustrate different aperture arrangements of a catheter;

FIG. 14 illustrates a securing mechanism in a left atrial appendage;

FIGS. 15a and 15b illustrate two exemplary securing mechanisms.

### DETAILED DESCRIPTION OF EMBODIMENT(S)

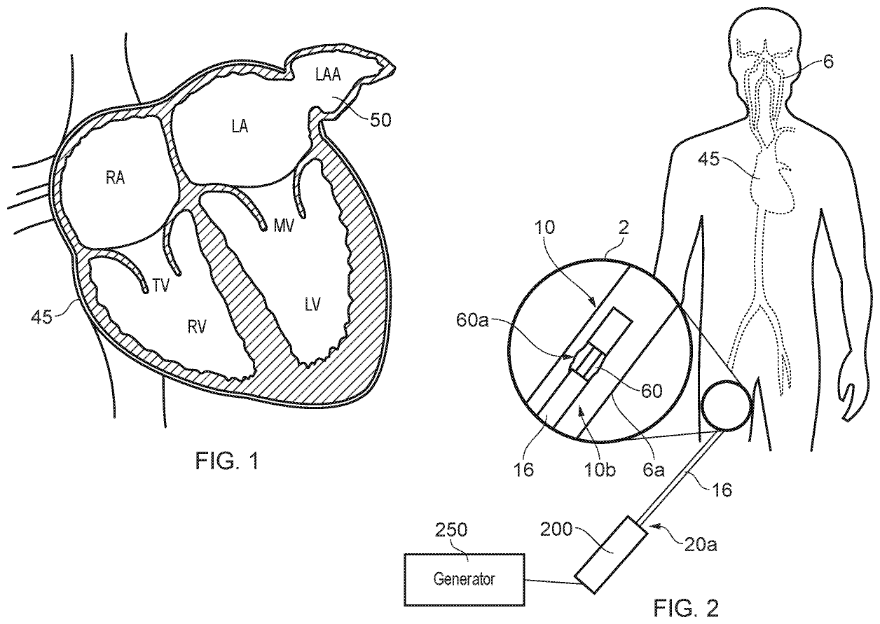

FIG. 1 is a schematic sectional illustration of a human heart 45 that depicts the four heart chambers (right atrium RA, right ventricle RV, left atrium LA, left ventricle LV) and a left atrial appendage LAA 50.

FIG. 2 illustrates a diagrammatic view of a patient undergoing an exemplary left atrial appendage occlusion procedure. As indicated by the enlarged circle area 2, an occlusion device 10 may be inserted into a vasculature 6 of a patient, including for example, into an artery 6a (e.g. a femoral artery) or vein (e.g., femoral vein, internal jugular vein). The occlusion device 10 includes a catheter 20 that is arranged to be positioned next to the left atrial appendage 50 within the heart 45. The occlusion device 10 is formed of a proximal portion (not shown) and a distal portion 10b. The proximal portion of the occlusion device 10 remains outside of the patient's body while the distal portion 10b is advanced through the patients' vasculature 6 towards the left atrial appendage 50.

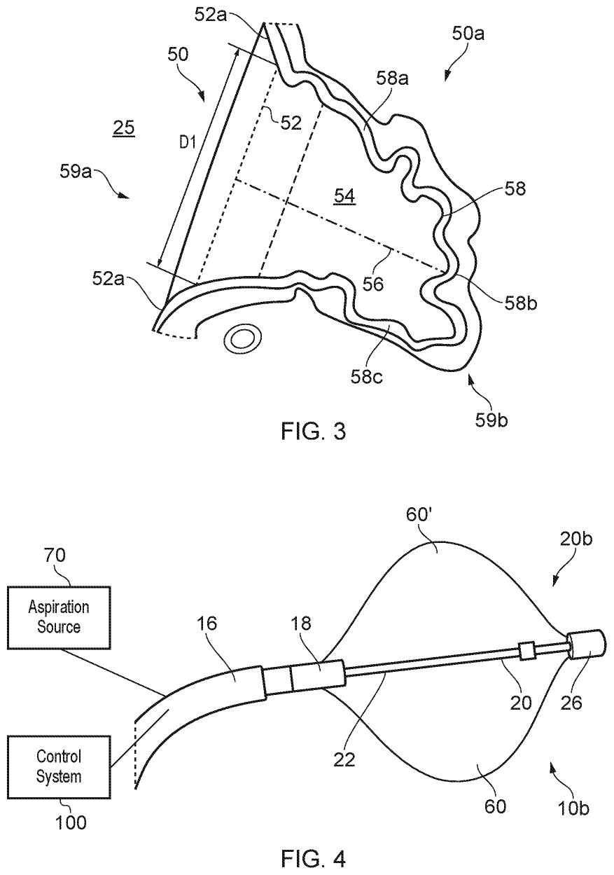

The occlusion device 10 is arranged to occlude (or obstruct) a left atrial appendage 50. While the shape of left atrial appendage 50 will vary for among patients, the left atrial appendage 50 is shown in FIG. 3 as being generally bursiform (pouch) shaped and has an opening 52 at a proximal end 59a that extends to a distal end 59b. The opening 52 is generally circular in shape and has a diameter D1. As shown, the opening 52 generally corresponds to the widest part of the left atrial appendage 50.

The left atrial appendage 50 has an internal volume 54 that is in fluid communication with the left atrium LA. The internal volume 54 may contain a volume of blood from the heart 45. The internal volume 54 is partially enclosed by a wall 58. As shown, the wall 58 has an irregular shape that generally forms an ear-shaped structure. In this example, the left atrial appendage wall 58 is formed of a superior wall 58a, an inferior wall 58c and a rear wall 58b. The left atrial appendage wall 58 includes further anterior and posterior wall sections that are not shown in FIG. 3.

The left atrial appendage 50 may have any type of morphology. For example, the left atrial appendage 50 may have a depth greater than the opening diameter D1, a smaller than the opening diameter D1, have a chicken-wing shape, cactus shape or cauliflower shape.

The left atrial appendage has a depth 56 that extends from the opening 52 (at the proximal end 59a) to the rear wall 58b (at the distal end 59b). Generally, the depth 56 of the left atrial appendage 50 is greatest at the rear wall 58b. The left atrial appendage 50 is in a natural, pre-treated state 50a in FIG. 3.

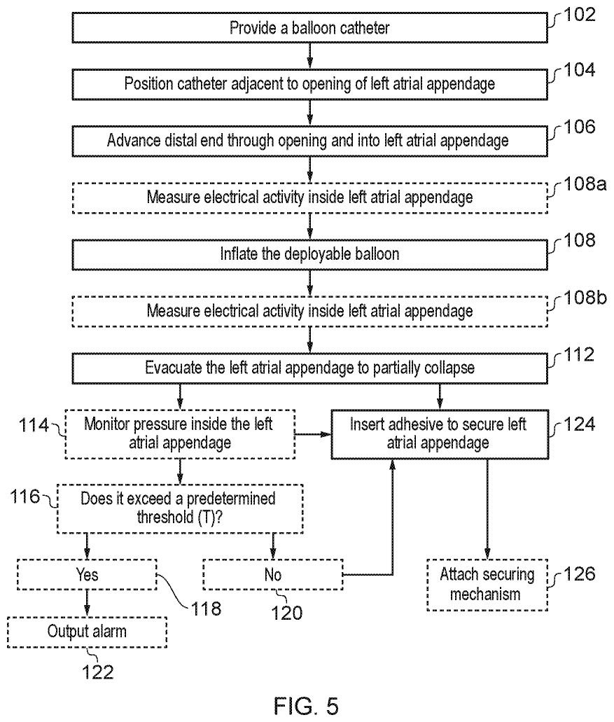

An exemplary catheter 20 and corresponding method for occluding a left atrial appendage is shown in FIGS. 4 and 5. As shown, the catheter 20 is housed in delivery catheters 16, 18 to form a nested arrangement. It will be understood that any suitable arrangement of catheters 16, 18, 20 may be used. For example, only two catheters (e.g. one delivery catheter 16, 18 and the catheter 20) may be provided, or a single catheter 20 may be used. The catheter 20 has an elongate tubular body 22 that extends from the proximal portion (not shown) to the distal portion 20b of the catheter 20. The catheter 20 has a central lumen 24 (shown more clearly in FIG. 10) that extends from a proximal end 20a of the catheter 20 to the distal end 20b of the catheter.

As shown schematically in FIG. 2, the proximal end 20a of the catheter and the deliver catheters 16, 18 are housed in a handle 200 of the occlusion device 10. The proximal end 20a of the catheter therefore remains outside of the patient's body. A medical practitioner manipulates the position and orientation of the catheter 20 by using the handle 200. As shown, the handle 200 may also be connected to any number of systems known to those skilled in the art, such as a generator 250.

As shown, the catheter 20 has an enlarged tip 26 at the distal end 20b of the catheter 20. The enlarged tip 26 provides stability to the catheter 20 as a medical practitioner manoeuvres the catheter 20 in the vasculature 6 of the patient or in the left atrial appendage 50. An end of a deployable member 60 (discussed further below) may be secured to the catheter 20 or may be secured to the enlarged tip 26. the other end of the deployable member 60 may be is secured to either delivery catheter 16, 18, or to the catheter 20. The deployable member 60 is arranged to be secured to the distal end 20b of the catheter 20. The deployable member 60 is therefore also housed inside the delivery catheters 16, 18.

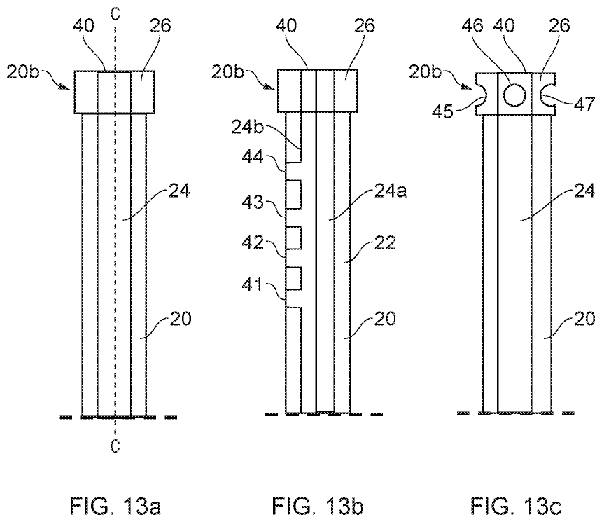

As shown more clearly in FIG. 13a, the central lumen 24 of the catheter 20 extends to an aperture 40, which in this example, is at the distal end of the enlarged tip 26. FIG. 12 shows the distal end 20b of exemplary catheter 20 that does not have an enlarged tip 26. In this example, the aperture 40 is located at the distal end 20b of the tubular body 22.

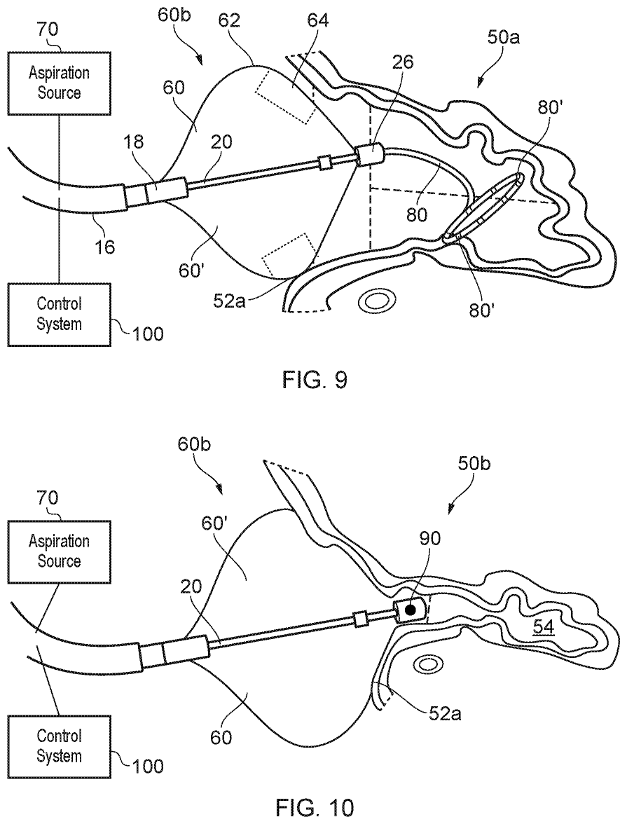

In this example, the deployable member 60 is a balloon 60′, however, other expandable devices may be used. As shown, the deployable member 60 (e.g., the balloon in this example) is arranged at the distal end 20b of the catheter 20. The deployable member 60 is arranged to move from a collapsed state 60a (shown in FIGS. 2, 6 and 8) to an inflated state 60b (shown in FIG. 9). The deployable member 60 is in a collapsed state 60a (i.e. not inflated) when the deployable member 60 is housed inside the delivery catheters 16, 18. The deployable member 60 is therefore in a collapsed state 60a as the catheter 20 is moved through the patient's vasculature 6 towards the left atrial appendage 50. The distal end 20b of the catheter 20 is arranged to be positioned adjacent to the opening 52 of the left atrial appendage 50.

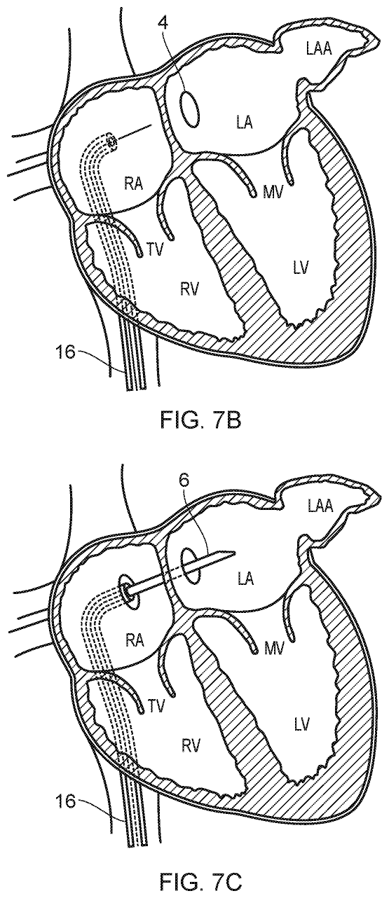

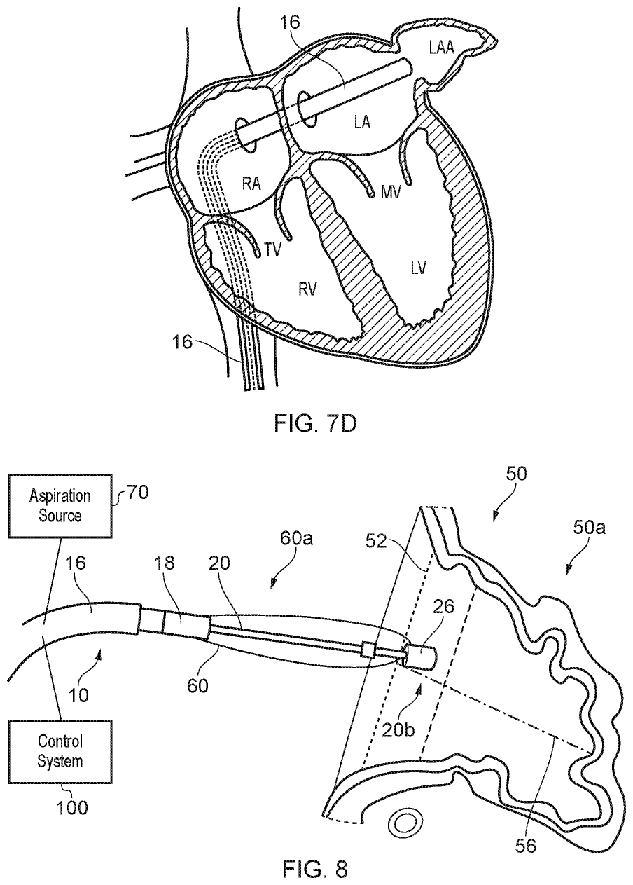

Referring to FIGS. 5 and 7A to 7D, a method for delivering and deploying an occlusion device to a left atrial appendage LAA will now be described. In an exemplary embodiment, a guidewire 2 is advanced after having been introduced into the vasculature via a percutaneous entry point and tracked through the vasculature into a left atrium LA of a heart. Intravascular access to the right atrium RA may be achieved via a percutaneous access site to femoral venous access up to the inferior venal cava, or other known access routes. Thereafter, a guidewire is advanced through the circulatory system, eventually arriving at the heart.

The guidewire 2 is directed into the right atrium RA (see FIG. 7A), traverses the right atrium and is made to traverse, with the aid of a pre-existing hole 4 (see FIG. 7B) or a transseptal needle 6 (see FIG. 7C) an atrial septum, thereby entering the left atrium LA. Once the guidewire 2 is positioned, the entry port and the atrial septum are dilated to permit entry of the delivery catheter 16 into the left atrium LA towards the left atrial appendage LAA (see FIG. 10D).

There, a deployable member 60 of the catheter 20 is positioned proximate the left atrial appendage LAA. Although described as a transfemoral antegrade approach for percutaneously accessing left atrium LA, the delivery catheter 16 may be positioned within the desired area of the heart via different methods or routes. For example, and not by way of limitation, another possible path would be through the radial vein into the brachial vein, through the subclavian vein, through the superior vena cava into the right atrium, and then transeptally into the left atrium.

Yet another possible path would be through the femoral artery into the aorta (as shown in FIG. 2), through the aortic valve into the left ventricle, and then retrograde through the mitral valve into the left atrium. In another embodiment, the left ventricle LV may be accessed via a transapical approach, and the delivery catheter 16 may be advanced through the left ventricle LV, the mitral valve, and into the left atrium LA adjacent the left atrial appendage LAA. In addition, although described with the use of a guidewire, in another embodiment hereof the delivery catheter 16 may access the left atrium LA without the use of a guidewire.

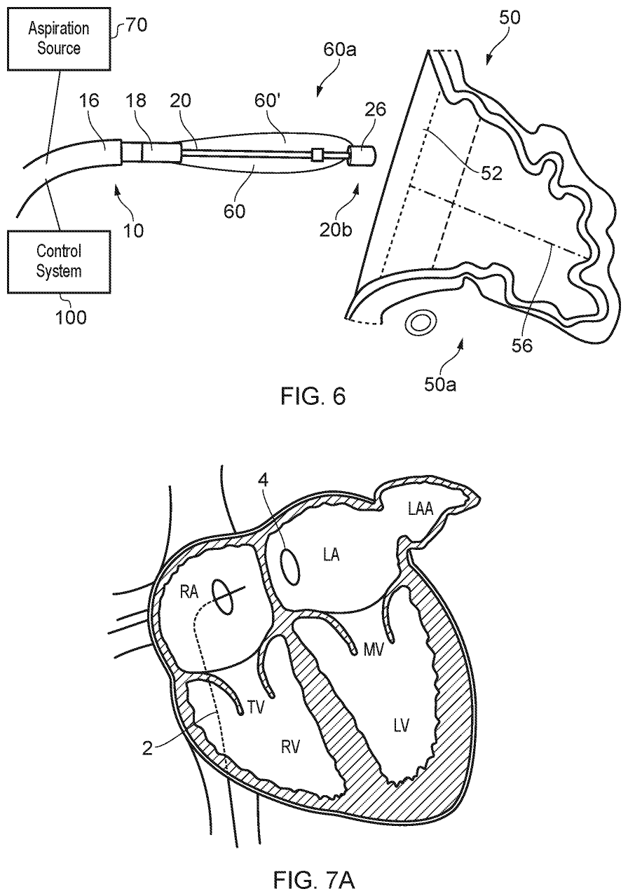

As shown in FIG. 6, the deployable member 60 remains in a collapsed state 60a when the catheter 20 is positioned near the opening 52 of the left atrial appendage 50. Once the distal end 20b of the catheter 20 is adjacent to the opening 52, the distal end 20b of the catheter 20 is advanced through the opening 52 and into the volume 54 of the left atrial appendage 50 at step 106. The enlarged tip 26 passes through the opening 52 so that the distal end 20b is positioned inside the internal volume 54 of the left atrial appendage 50.

When fully inflated, an outer surface 62 of the deployable member 60 contacts an area 52a surrounding the opening 52 as shown in FIG. 9. The deployable member 60 contacts the area 52a around the opening 52 so that the deployable member 60 creates a seal between the deployable member 60 and an internal volume 54 of the left atrial appendage 50. When the deployable member 60 is inflated, the left atrial appendage 50 is no longer in fluid communication with the left atrium 25. Inflating the deployable member 60 to form a seal creates an internal pressure within the left atrial appendage 50. When the deployable member 60 is a balloon 60′, the balloon 60′ is generally formed of easily deformable material. The outer surface 62 of the balloon 60′ therefore easily deforms and adjusts to the shape of the area 52a surrounding the opening 52. The balloon 60′ is therefore physically compliant against the area 52a surrounding the opening 52 because the balloon 60′ conforms to the shape of the area 52a.

Preferably, the balloon 60′ is a cryoballoon. In a fully inflated state 60b, the balloon 60′ may contain gas with a volume of cryogenic fluid subsequently injected into the balloon, or may be fully filled with cryogenic fluid (cryofluid) or refrigerant. In this example, when cryogenic fluid/refrigerant is introduced into the balloon, the outer surface 62 of the balloon cryoadheres to the area 52a surrounding the opening 52. The outer surface 62 of the cryoballoon may have a temperature between 0 and −15° C. This ensures that the outer surface 62 may cryoadhere to the area 52a surrounding the opening 52. However, the outer surface 62 of the cryoballoon may be at any suitable temperature to achieve cryoadhesion between the cryoballoon and the tissue 52a.

The cryoadherance of the cryoballoon 60 ensures that a seal is easily maintained between the balloon 60 and the left atrial appendage 50. The cryoballoon ensures that a seal is maintained throughout the procedure more easily than a normal balloon, which typically relies on having continuous mechanical contact against the area 52a.

To easily remove the cryoballoon 60′ from the area 52a, the outer surface of the balloon 60′ may be modified so that a portion 64 of the outer surface 62 is formed from a different material than the rest of the outer surface 62. When deflated, (i.e. the cryogenic fluid is removed from the balloon 60′), the cryoadhered portions 64 are more likely to detach from the tissue at the area 52a. The portion(s) 64 may be formed from a material such as polytetrafluoroethylene (PTFE). As shown, the portion(s) 64 may be positioned proximally to the distal end 20b of the catheter 20 so that when the balloon 60′ is in the inflated state 60b, the portion(s) 64 contacts the area 52a. Optionally, the portions 64 may be arranged to detach from the rest of the balloon 60′ so that after the portions 64 contact the area 52a, they remain attached to the area 52a after the balloon 60′ is deflated and the catheter 20 is removed from the patient.

The catheter 20 may optionally include an electrical sensor 80 as shown in FIG. 9. In this example, the electrical sensor 80 is lasso shaped arrangement of a plurality of electrodes and is arranged to be extended into the left atrial appendage 50 by a medical practitioner. In other instances, however, electrical sensor 80 may include one or more electrodes taking any of a number of shapes. The electrical sensor 80 is housed within the catheter 20, e.g., inside the lumen 24 of the catheter 20, and extends and retracts from catheter 20.

In other examples, the electrical sensor 80 may be positioned on the distal end 20b of the catheter 20, for example, on the tubular body 22 or on the enlarged tip 26. The electrical sensor 80 may also be introduced into the left atrial appendage 50 by another catheter. In this example, the electrical sensor 80 is arranged to be positioned between the deployable member 60 and the area 52a of the left atrial appendage 50. Preferably, the electrical sensor 80 does not affect the seal created between the deployable member 60 and the left atrial appendage 50. The electrical sensor 80 is arranged to obtain an electrical measurement corresponding to local cardiac activation (normally transmitted from the sinoatrial node (SA node) throughout the rest of the heart 45) inside the left atrial appendage 50. As shown in FIG. 5, the electrical reading may be obtained before and after the balloon 60 is inflated. Alternatively, the electrical sensor 80 may be arranged to measure local impedance of the left atrial appendage 50, which may alter when blood is removed from the internal volume 54 of the left atrial appendage 50, or when adhesive is introduced into the left atrial appendage 50,

When in the inflated state 60b, the deployable member 60 creates a seal between the deployable member 60 and the left atrial appendage 50 to form an internal pressure in the volume 54 of the left atrial appendage 50. The catheter 20 partially evacuates the left atrial appendage 50 by aspirating the internal volume 54 of the left atrial appendage (using an aspiration source 70). The internal volume 52 may include a volume of blood or air, so the aspiration source 70 may be used to remove these from inside the left atrial appendage 50.

The aspiration source 70 is in fluid communication with the central lumen 24 of the catheter 20. Therefore, the aspiration source 70 is operable to aspirate a location at the distal end 20b of the catheter 20 through the aperture 40. Aspirating the volume 54 of the appendage 50 creates a reduced or negative pressure in the left atrial appendage 50, which causes the walls 58 of the left atrial appendage 50 to be drawn closer together. As the walls 58 are drawn closer together, the volume 54 of the left atrial appendage 50 also reduces.

As shown in FIG. 10, in a partially collapsed state 50b, the distance between the walls 58a-58c is less than when the left atrial appendage 50 is in a natural pre-treated state 50a. In this example, the left atrial appendage 50 is collapsed to fit the width of the enlarged tip 26. In other examples, the left atrial appendage 50 may only be partially collapsed so the internal volume 54 only reduces by a small percentage of the original volume 54, or until the internal volume 54 reaches a specific threshold that is observable by a medical practitioner using additional imaging.

Preferably, the left atrial appendage 50 is collapsed so that the walls 58a-58c of the left atrial appendage contact. This minimises the volume of adhesive necessary to hold the left atrial appendage in a collapsed state 50b. This also reduces the risk of the walls 58a-58c detaching or causing an embolism. Alternatively, the left atrial appendage 50 may be collapsed until a negative pressure is observed in the left atrial appendage 50. This ensures that any adhesive that is subsequently injected into the internal volume 54 will remain inside the left atrial appendage 50. The catheter 20 preferably has at least one aperture 40 at the distal end 20b of the catheter 20. However, the catheter 20 may have any number of apertures and arrangements to evacuate the left atrial appendage 50. FIGS. 13a-13c show different aperture arrangements for a catheter 20. FIGS. 13a-13c show horizontal cross sections along line A-A of the catheter 20 shown in FIG. 12. In the first example shown in FIG. 13a, the catheter 20 has one aperture 40 located at the distal end 20b of the catheter. As shown, the aperture 40 is in fluid communication with the lumen 24.

FIG. 13b shows a second exemplary aperture arrangement. In this example, the catheter 20 is a multi-lumen catheter, and has a first lumen 24a in fluid communication with only aperture 40, a second lumen 24b in communication with the apertures 40-44. The aperture 40 is located at the distal end 20b of the catheter 20 and is on the enlarged tip 26. In this example, the catheter 20 further includes apertures 41, 42, 43, 44 that are positioned on the tubular body 22 of the catheter 20. As shown, the apertures 41-44 are arranged along the longitudinal length of the catheter 20.

The position of the apertures 41-44 may be moved if the deployable member 60 is arranged to be secured to the enlarged tip 26. If the deployable member 60 is arranged to be secured on to the enlarged tip 26, a further catheter (not shown) may be inserted into the left atrial appendage 50 through the first lumen 24a. The apertures 41-44 may be arranged on the further catheter so that the medical practitioner may introduce adhesive into the left atrial appendage 50 using the further catheter.

In the arrangement shown in FIG. 13B, the distal aperture 40 and the apertures 41-44 may be used for different purposes. For example, the distal aperture 40 may be used for evacuating the left atrial appendage 50 through the first lumen 24a. The distal aperture 40 and the apertures 41-44 may be used to introduce adhesive into the left atrial appendage 50 through the second lumen 24b. The adhesive may be introduced into the left atrial appendage 50 after evacuating the left atrial appendage 50 or may be introduced as the left atrial appendage 50 is evacuated. In another example, the catheter 20 may have a third lumen (not shown) that is in fluid communication with an aperture (e.g. aperture 41) so that cryofluid may be introduced into an internal volume of the deployable member 60.

The size of the apertures 40-44 may be modified depending on the function of the aperture. For example, the aperture 40 may be sized to ensure that blood easily enters the aperture 40 but restricts the entry of adhesive through the aperture 40.

Alternatively, the apertures 40-44 may all be used to evacuate the left atrial appendage 50 before introducing adhesive. Using multiple apertures 40-44 ensures that at least one aperture 40-44 remains in fluid communication with the left atrial appendage in case of obstruction.

The apertures 41-44 extend perpendicularly from a central axis C of the lumen 24. In this example, there are only four apertures along the tubular body 22. However, any number of apertures may be positioned along the tubular body 22 and in fluid communication with the lumen 24. Similarly, while only four apertures are shown on one side of the catheter 20, it will be understood that the apertures may be positioned on either side of the tubular body 22 in any suitable arrangement.

FIG. 13c shows a third exemplary aperture arrangement. Similar to the first and second aperture arrangements shown in FIGS. 13a and 13b, the catheter 20 includes an aperture 40 located on the enlarged tip 26. In this example, the distal end 20b further includes at least one further aperture arranged in a radial arrangement around a circumference of the enlarged tip 26. In this example, the catheter 20 has 6 apertures (3 of which are visible in FIG. 13 c). As shown, the apertures 45, 46, 47 are arranged in a radial arrangement around the enlarged tip 26. The aspiration source 70 is operable to at least partially evacuate the left atrial appendage 50 through the plurality of apertures 40-47. The different aperture arrangements shown in FIGS. 13b and 13c may be used to aspirate the left atrial appendage 50 in case any of the apertures become partially or fully obstructed, for example, by blood clots or by contacting the tissue inside the left atrial appendage 50.

The catheter 20 may optionally include a pressure sensor 90 as shown in FIG. 10. In this example, the pressure sensor 90 is shown schematically but may be positioned at any suitable position at the distal end 20b of the catheter 20. Alternatively, the pressure sensor 90 may be provided on another catheter through the lumen 24 of the catheter 20 so that it extends into the left atrial appendage 50 by a medical practitioner.

The pressure sensor 90 is arranged to measure the pressure inside the left atrial appendage 50. The pressure sensor 90 is electrically connected to the control system 100 and communicates the measured pressure reading to the control system 100. The pressure sensor 90 may continuously measure the pressure inside the left atrial appendage 50 as the left atrial appendage is evacuated or may optionally measure pressure after the left atrial appendage 50 has been evacuated and is in a collapsed state 50b. The measured pressure is communicated to the control system 100 which establishes whether or not the measured pressure crosses a predetermined threshold, T. The predetermined threshold T may be indicative of the aperture(s) 40 being clogged (for example, with thromboembolic matter), or that the distal end 20b of the catheter 20 is contacting the left atrial appendage wall 58.

If the measured pressure does cross the predetermined threshold T, the control system 100 is arranged to emit an alarm to alert the medical practitioner. The medical practitioner may then stop the aspiration source 70 to free the aperture(s) of the catheter 20 or may move the distal end 20b in the left atrial appendage to try and free the obstruction in the catheter aperture(s) or lumen 24. Alternatively, the system 100 may be arranged to stop the aspiration source 70 if the measured pressure crosses the predetermined threshold T. The control system 100 may use the measured pressure as a feedback loop to control the aspiration source 70. For example, the control system 100 may adjust the vacuum provided by the aspiration source 70 depending on the measured pressure. This may be used to optimise emptying (for example, start at a high vacuum and reduce the vacuum as the apertures become partially blocked or when they contact the tissue), and may be reduced when the aspiration source 70 is used to keep the left atrial appendage 50 in the collapsed state 50b. The pressure in the left atrial appendage 50 may be measured until the left atrial appendage 50 is in a collapsed state 50b. If the measured pressure does not exceed a predetermined threshold T during evacuating the left atrial appendage 50, the medical practitioner may then proceed to secure the left atrial appendage 50 in the collapsed state 50b.

Adhesive 30 is delivered into the partially collapsed atrial appendage 50b to secure the left atrial appendage 50 in the partially collapsed state 50b. The adhesive 30 is preferably delivered to the left atrial appendage 50 through a steerable delivery catheter 35. As shown more clearly in FIG. 12, the steerable delivery catheter 35 has a central lumen 36 that extends from a proximal end to an opening 37 at the distal end of the delivery catheter 35 (not shown).

The steerable delivery catheter 35 may be provided outside of the catheter 20. For example, the steerable delivery catheter 35 may be manoeuvred to the left atrial appendage 50 by the medical practitioner and between the deployable member 60 and the area 52a so that the delivery catheter 35 may insert adhesive 35 into the left atrial appendage 50.

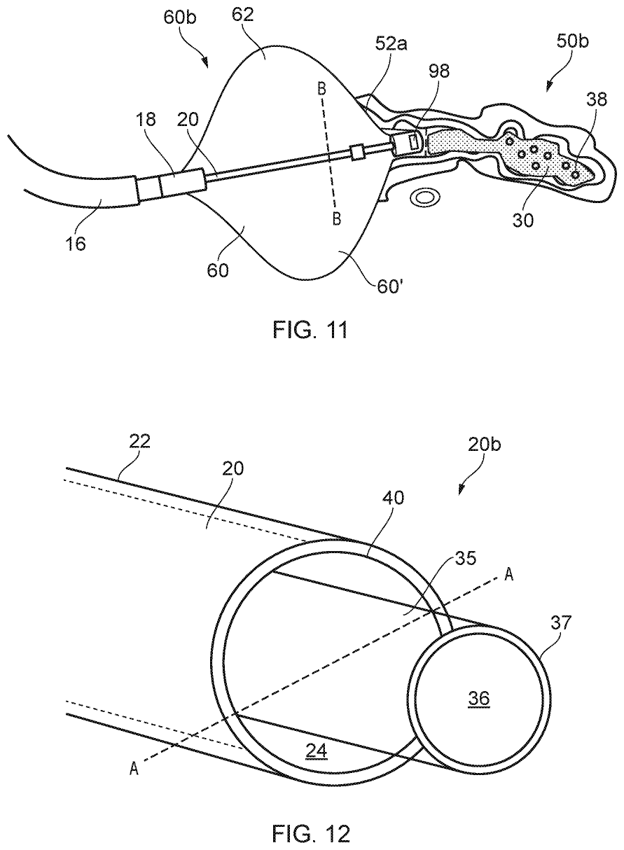

Preferably, the steerable delivery catheter 35 is housed in the lumen 24 of the catheter 20. By providing the steerable delivery catheter 35 inside the catheter 20, it ensures that the seal created between the deployable member 60 and the area 52a is not disrupted. FIG. 12 shows a cross section of an exemplary catheter 20 along lines B-B in FIG. 11.

The adhesive 30 is inserted into the volume 54 of the left atrial appendage 50 in the partially collapsed state 50b. The adhesive 30 contacts the walls 58a-58c to secure the left atrial appendage 50 in the partially collapsed state 50b. Once secured, the left atrial appendage 50 does not return to the natural, pre-treatment state 50a. The adhesive 30 may be any suitable bioglue, such as cyanoacrylate-based adhesive, bovine serum albumin-glutaraldehyde-based, hydrogel based or animal-inspired adhesive. Preferably, the bioglue is any suitable medically inert substance that is arranged to adhere to tissue, such as the LAA walls. The adhesive 30 may be incorporated with radiopaque markers 38 (shown schematically in FIG. 11) so that a medical practitioner may observe the bioglue using conventional imaging techniques, such as fluoroscopy.

Preferably, the adhesive 30 is inserted and cured before the seal formed between the left atrial appendage 50 and the deployable member 60 is removed. The adhesive 30 may be arranged to cure after a period of time, or by using a curing agent, such as a light source 98. Preferably, the light source 98 is arranged at the distal end 20b of the catheter 20, but may be provided on a further catheter (not shown) that is inserted into the left atrial appendage 50. FIG. 11 shows an exemplary light source 98 on the enlarged tip 26 of the catheter 20. In other examples, when the deployable member 60 is formed of a transparent material, the light source 98 may be positioned within the deployable member 60. The light source 98 may, for example, be an ultraviolet (UV) light source or visible light source.

When the deployable member 60 is a cryoballoon, the outer surface 62 of the cryoballoon may be used to cryoablate the tissue in the area 52a surrounding the opening 52 after the adhesive 30 has been inserted into the internal volume 54 of the left atrial appendage. The outer surface 62 of the cryoballoon may have a temperature between −40 and −50° C. However, the outer surface 62 of the cryoballoon may be at any suitable temperature to achieve cryoablation between the cryoballoon and the tissue 52a. Cryoablating the area 52a ensures stability of the tissue in the area 52a and also ensures that the walls 58a-58c remain adhered together after the cryoballoon is removed.

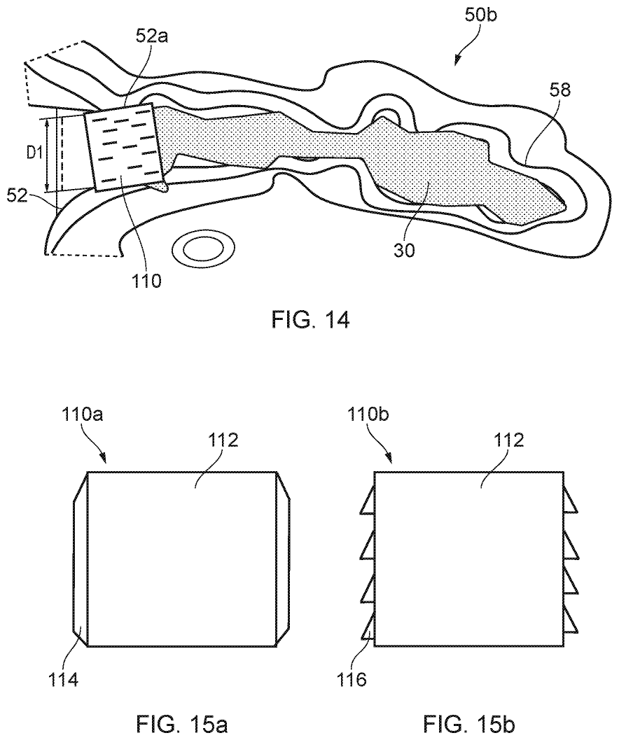

The adhesive 30 secures the left atrial appendage 50 in an occluded state by adhering the walls 58a-58c together. Optionally, the catheter 20 may further include a securing mechanism 110 that is arranged to mechanically engage with the left atrial appendage 50 to hold it in a collapsed state 50b (as shown in FIG. 14).

The diameter D1 of the opening 52 in a collapsed state 50b is smaller than the diameter D1 of the opening in a natural, pre-treated state 50a. The walls and tissue that form the area 52a are therefore closer together in the collapsed state 50b. The securing mechanism 110 is arranged to engage with the area 52a surrounding the opening 52 to prevent the opening 52 from expanding back to the natural, pre-treated state 50a.

FIGS. 15a and 15b show two cross-sectional views of exemplary securing mechanisms 110a, 110b. As shown in FIG. 15a, the securing mechanism 110a includes a generally tubular body 112 with a plurality of tines 114 that extend from the body 112. Similarly, the securing mechanism 110b has a generally tubular body 112 with a plurality of barbs 116 that extend from the body 112. The tines 114 and barbs 116 extend from the tubular body 112 to either contact and/or partially embed into the tissue around the left atrial appendage opening 52. The securing mechanism 110 may include any combination of tines 114 or barbs 116. Preferably, the securing mechanism 100 contacts the tissue surrounding the opening 52. Preferably, the securing mechanism 100 contacts the area 52a on at least opposing sides of the opening 52 to hold the opening 52 at a distance D1. The securing mechanism 110 therefore acts as an anchor that maintains the distance D1 of the opening 52. Alternatively, the securing mechanism 110 may be a staple that is secured to either side of the area 52a to secure the opening 52 closed.

The securing mechanism 110 may be deployed through a second catheter (not shown) that is positioned proximal to the opening 52 of the left atrial appendage 50 after the adhesive 30 has been cured. Alternatively, the securing mechanism 110 may be detachable from the first catheter 20. For example, the securing mechanism 110 may form part of the enlarged tip 26 or may form part of the tubular body 22 which may be selectively detached from the first catheter 20 after the adhesive 30 has cured. The securing mechanism 110 may be selectively detached by using a detachment mechanism (not shown). The detachment mechanism may include a looped wire that extends around the enlarged tip 26 and through the central lumen 24 of the catheter 20. Once the securing mechanism 110 is secured, the practitioner may pull one strand of the loop to release the securing mechanism 110 from the enlarged tip 26. Alternatively, the securing mechanism 110 may be screwed into the enlarged tip 26, so that the securing mechanism can be selectively unscrewed from the tip 26. The medical practitioner may therefore occlude the left atrial appendage 50 without having to introduce different equipment into the patient.

An exemplary occlusion device 10 for occluding a left atrial appendage 50 is described above. An exemplary method for occluding the left atrial appendage 50 is described below in relation to the flow chart shown in FIG. 5.

At step 102, a catheter 20 is provided. Once the distal end 20b of the catheter 20 is adjacent to the opening at step 104, the distal end 20b of the catheter 20 is advanced through the opening 52 towards the distal end 59b of the left atrial appendage 50. As shown in FIG. 8, the distal end 20b of the catheter is positioned into the volume 54 of the left atrial appendage at step 106.

If the deployable member 60 is a balloon 60′, the deployable member 60 is preferably expanded (i.e. in the inflated state 60b) at step 108. Optionally, if the catheter includes an electrical sensor 80, an electrical reading is obtained at step 108a. The electrical reading obtained at step 108a provides a first measurement that indicates the electrical activity observed inside the left atrial appendage 50 before the balloon 60′ cryoadheres to the area 52a. The first measurement is communicated with the control system 100.

At step 108b, the electrical sensor 80 measures the electrical activity inside the left atrial appendage after the balloon 60 is inflated and cryoablates the tissue in the area 52a to obtain a second measurement. If the balloon 60′ has effectively cryoadhered to form a seal between the balloon 60′ and the left atrial appendage 50, the second measurement will be less than the first measurement. The seal may also be verified by other imaging techniques, such as injecting radiopaque contrast agent under fluoroscopy or intracardiac echography. The second measurement is communicated with the control system 100. The first and second measurements may be used by the medical practitioner to determine whether the balloon 60′ is in the correct position before proceeding on to evacuating the left atrial appendage 50 at step 112.

After the balloon 60′ has been inflated at step 108 and positioned against the opening 52, the medical practitioner proceeds to at least partially evacuate the left atrial appendage 50 at step 112, as shown in FIG. 10.

If the occlusion device 10 includes a pressure sensor 90, the pressure inside the left atrial appendage 50 may be obtained at step 114 during or after evacuating the left atrial appendage 50 at step 112. If the catheter 20 does not have a pressure sensor 90, then the medical practitioner may move on to step 124 to insert adhesive 30 into the left atrial appendage 50.

If the measured pressure does cross the predetermined threshold T at step 118, the control system 100 emits an alarm at step 122 to alert the medical practitioner. The medical practitioner may then stop the aspiration source 70 to free the aperture(s) of the catheter 20, or may move the distal end 20b in the left atrial appendage to try and free the obstruction in the catheter aperture(s) or lumen 24.

The pressure in the left atrial appendage 50 may be measured until the left atrial appendage 50 is in a collapsed state 50b. If the measured pressure does not exceed a predetermined threshold T during evacuating the left atrial appendage 50 at step 120, the medical practitioner may then proceed to step 124 to secure the left atrial appendage 50 in the collapsed state 50b.

At step 124, adhesive 30 is inserted into the partially collapsed atrial appendage 50b to secure the left atrial appendage 50 in the partially collapsed state 50b. The adhesive 30 is preferably delivered to the left atrial appendage 50 through a steerable delivery catheter 35. As shown more clearly in FIG. 12, the steerable delivery catheter 35 has a central lumen 36 that extends from a proximal end to an opening 37 at the distal end of the delivery catheter 35 (not shown).

If the occlusion device 10 has a securing mechanism 110, the medical practitioner may optionally attach the securing mechanism 110 to the left atrial appendage 50 at step 126 after adhesive has been delivered at step 124. Once the adhesive 30 is cured, the balloon 60′ returns to a collapsed state 60a to remove the seal between the balloon 60′ and the left atrial appendage 50. The catheter 20 is then withdrawn from the heart 45 with the left atrial appendage 50 secured in the partially collapsed state 50b.

With regard to the terms “distal” and “proximal” within this description, unless otherwise specified, the terms can reference a relative position of the portions of the delivery catheter system with reference to a medical practitioner and/or a location in the vasculature or heart. For example, “proximal” can refer to a position closer to the medical practitioner of the device or an incision into the vasculature, and “distal” can refer to a position that is more distant from the medical practitioner of the device or further from the incision along the vasculature (e.g., the end of the catheter).

Although the teachings have been described above with reference to one or more preferred embodiments, it will be appreciated that various changes or modifications may be made without departing from the scope as defined in the appended claims.

Where the word ‘or’ appears this is to be construed to mean ‘and/or’ such that items referred to are not necessarily mutually exclusive and may be used in any appropriate combination. Although the invention has been described above with reference to one or more preferred embodiments, it will be appreciated that various changes or modifications may be made without departing from the scope of the invention as defined in the appended claims.

### CLAUSES

Clause 1. A method of occluding a left atrial appendage, the method comprising:
providing a catheter with an elongate tubular body and a central lumen that extends from a proximal end to a distal end of the catheter, the catheter comprising a deployable balloon arranged at the distal end of the catheter;
positioning the catheter adjacent to an opening of the left atrial appendage;
advancing the distal end of the catheter through the opening and into the left atrial appendage;
inflating the balloon to contact an area surrounding the opening of the left atrial appendage to create a seal between the balloon and the left atrial appendage;
evacuating the left atrial appendage to at least partially collapse the left atrial appendage; and
inserting adhesive into the at least partially collapsed left atrial appendage to secure the left atrial appendage in the at least partially collapsed state.

Clause 2. The method of occluding the left atrial appendage according to clause 1, wherein the left atrial appendage has a pressure after the balloon contacts an area surrounding the opening of the left atrial appendage, and
wherein evacuating the left atrial appendage comprises creating a reduced pressure in the left atrial appendage to reduce a volume of the left atrial appendage.

Clause 3, The method of occluding the left atrial appendage according to clause 1 or 2, wherein evacuating the left atrial appendage comprises aspirating through at least one aperture at the distal end of the catheter to create the reduced pressure in the left atrial appendage.

Clause 4. The method of occluding the left atrial appendage according to any preceding clause, wherein the catheter is a cryoballoon catheter and fully inflating the balloon comprises at least partially filling the balloon with cryofluid or refrigerant to contact the area surrounding the opening of the left atrial appendage.

Clause 5. The method of occluding a left atrial appendage according to clause 4, wherein the cryoballoon is collapsed when the catheter is positioned adjacent to the opening of the left atrial appendage.

Clause 6. The method of occluding the left atrial appendage according to clause 4 or 5, wherein the catheter further comprises an electrical sensor, and the method further comprises deploying the electrical sensor into the left atrial appendage and obtaining an electrical activity reading before and after the cryoballoon cryoadheres to the area surrounding the opening of the left atrial appendage.

Clause 7. The method of occluding the left atrial appendage according to any preceding clause, wherein the catheter further comprises a pressure sensor at the distal end of the catheter to measure the pressure in left atrial appendage and the method further comprises:
observing a pressure reading at the distal end of the catheter and
outputting an alarm if the observed pressure crosses a predetermined threshold.

Clause 8. The method of occluding the left atrial appendage according to any preceding clause, the method comprising incorporating radiopaque markers into the adhesive.

Clause 9. The method of occluding the left atrial appendage according to any preceding clause, wherein the method further comprises curing the adhesive before collapsing the balloon to remove the seal between the balloon and the left atrial appendage.

Clause 10. The method of occluding the left atrial appendage according to any preceding clause, wherein the adhesive is visible light or ultraviolet cured (UV) adhesive, wherein the method further comprises using a light source to cure the adhesive after the adhesive is inserted into the at least partially collapsed left atrial appendage.

Clause 11. The method of occluding the left atrial appendage according to clause 10, wherein the catheter further comprises the light source or wherein the method further comprises providing a further catheter comprising the light source.

Clause 12. The method of occluding the left atrial appendage according to any preceding clause, wherein the catheter further comprises a securing mechanism arranged to engage with an area surrounding the opening of the left atrial appendage, the method further comprising deploying the securing mechanism to engage with the area surrounding the opening of the left atrial appendage to secure the left atrial appendage in the at least partially collapsed state.

Clause 13. The method of occluding the left atrial appendage according to clause 12, wherein a portion of the distal end of the catheter is detachable and forms the securing mechanism, the method further comprising detaching the portion of the distal end of the catheter and engaging the detached portion with the area surrounding the opening of the left atrial appendage to secure the left atrial appendage in the at least partially collapsed state.

Clause 14. The method of occluding the left atrial appendage according to any one of clauses 1-11, the method further comprising:
providing a second catheter comprising a securing mechanism arranged to engage with an area surrounding the opening of the left atrial appendage; and
inserting a distal end of the second catheter towards the opening of the left atrial appendage before deploying the securing mechanism to engage with the area surrounding the opening of the left atrial appendage to secure the left atrial appendage in the at least partially collapsed state.

Clause 15. An occlusion device for occluding a left atrial appendage, the device comprising:
a first catheter with an elongate tubular body and a central lumen that extends from a proximal end to a distal end of the catheter, the distal end arranged to be positioned at an opening of the left atrial appendage;
a deployable balloon arranged at the distal end of the catheter,
wherein the deployable balloon is operable to seal against an opening of the left atrial appendage;
a steerable delivery catheter arranged to deliver adhesive at the distal end of the catheter; and
an aspiration source attached to the central lumen of the catheter, wherein the aspiration source is operable to at least partially evacuate the left atrial appendage to at least partially collapse the left atrial appendage.

Clause 16. The occlusion device according to clause 15, wherein the steerable delivery catheter is housed in the central lumen of the first catheter.

Clause 17. The occlusion device according to clause 15 or 16, wherein the deployable balloon is a cryoballoon

Clause 18. The occlusion device according to clause 17, wherein at least a portion of an outer surface of the cryoballoon is formed of a different material to the remaining outer surface of the cryoballoon.

Clause 19. The occlusion device according to any one of clauses 15 to 18, wherein the first catheter further comprises at least one aperture at the distal end of the first catheter, wherein the at least one aperture extends perpendicularly from the central lumen so that the aspiration source is operable to at least partially evacuate the left atrial appendage through the at least one aperture.

Clause 20. The occlusion device according to clause 19, wherein the first catheter further comprises a plurality of apertures along a longitudinal length of the first catheter, wherein the plurality of apertures extend perpendicularly from the central lumen, so that the aspiration source is operable to at least partially evacuate the left atrial appendage through the plurality of apertures.

Clause 21. The occlusion device according to clause 19, wherein the first catheter comprises at least two apertures in a radial arrangement around a circumference of the tubular body at the distal end of the first catheter so that the aspiration source is operable to at least partially evacuate the left atrial appendage through the at least two apertures.

Clause 22. The occlusion device according to any one of clauses 15 to 21, wherein the occlusion device further comprises a pressure sensor at a distal end of the first catheter, wherein the pressure sensor is arranged to measure pressure in the left atrial appendage, and optionally, the pressure sensor is arranged to send a signal to a control system if the pressure crosses a predetermined threshold.

Clause 23. The occlusion device according to any one of clauses 15 to 22, wherein the occlusion device further comprises a source that is arranged to cure the adhesive.

Clause 24. The occlusion device according to any one of clauses 15 to 23, wherein the adhesive is bioglue.

Clause 25. The occlusion device according to any one of clauses 15 to 24, wherein the occlusion device further comprises a deployable electrical sensor that is arranged to extend into a left atrial appendage to measure electrical activity in the left atrial appendage.

Clause 26. The occlusion device according to any one of clauses 15 to 25, wherein the occlusion device further comprises a securing mechanism, wherein the securing mechanism is arranged to engage with an area surrounding the opening of the left atrial appendage to secure the left atrial appendage in the at least partially collapsed state.

Clause 27. The occlusion device according to clause 26, wherein the securing mechanism forms part of the elongate tubular body of the first catheter, and wherein the securing mechanism is detachable from the first catheter.

Clause 28. The occlusion device according to any one of clauses 15 to 25, wherein the occlusion device further comprises a second catheter comprising a securing mechanism arranged to engage with an area surrounding the opening of the left atrial appendage; and preferably wherein the securing mechanism is detachable from the second catheter.

Clause 29. The occlusion device according to any one of clauses 26 to 28, wherein the securing mechanism comprises a plurality of tines and/or barbs, wherein the tines and/or barbs are arranged to mechanically engage with an area surrounding the opening of the left atrial appendage to secure the left atrial appendage in the at least partially collapsed state.

1. An occlusion device for occluding a left atrial appendage, the device comprising:
a first catheter with an elongate tubular body and a central lumen that extends from a proximal end to a distal end of the catheter, the distal end arranged to be positioned at an opening of the left atrial appendage;
a deployable balloon arranged at the distal end of the catheter,
wherein the deployable balloon is operable to seal against an opening of the left atrial appendage;
a steerable delivery catheter arranged to deliver adhesive at the distal end of the catheter; and
an aspiration source attached to the central lumen of the catheter, wherein the aspiration source is operable to at least partially evacuate the left atrial appendage to at least partially collapse the left atrial appendage.

2. The occlusion device according to claim 1, wherein the steerable delivery catheter is housed in the central lumen of the first catheter.

3. The occlusion device according to claim 1, wherein the deployable balloon is a cryoballoon.

4. (canceled)

5. The occlusion device according to claim 1, wherein the first catheter further comprises at least one aperture at the distal end of the first catheter, and
wherein the at least one aperture extends perpendicularly from the central lumen so that the aspiration source is operable to at least partially evacuate the left atrial appendage through the at least one aperture.

6. The occlusion device according to claim 5, wherein the first catheter further comprises a plurality of apertures along a longitudinal length of the first catheter, and
wherein the plurality of apertures extend perpendicularly from the central lumen, so that the aspiration source is operable to at least partially evacuate the left atrial appendage through the plurality of apertures.

7. (canceled)

8. The occlusion device according to claim 1, wherein the occlusion device further comprises a pressure sensor at a distal end of the first catheter, and wherein the pressure sensor is arranged to measure pressure in the left atrial appendage.

9. The occlusion device according to claim 1, wherein the occlusion device further comprises a source that is arranged to cure the adhesive.

10. The occlusion device according to claim 1, wherein the adhesive is bioglue.

11. The occlusion device claim 1, wherein the occlusion device further comprises a deployable electrical sensor that is arranged to extend into a left atrial appendage to measure electrical activity in the left atrial appendage.

12. The occlusion device according to claim 1, wherein the occlusion device further comprises a securing mechanism, wherein the securing mechanism is arranged to engage with an area surrounding the opening of the left atrial appendage to secure the left atrial appendage in the at least partially collapsed state.

13. (canceled)

14. The occlusion device according to claim 1, wherein the occlusion device further comprises a second catheter comprising a securing mechanism arranged to engage with an area surrounding the opening of the left atrial appendage; and preferably wherein the securing mechanism is detachable from the second catheter.

15. (canceled)

16. A method of occluding a left atrial appendage, the method comprising:
positioning a catheter adjacent to an opening of the left atrial appendage, the catheter comprising an elongate tubular body and a central lumen that extends from a proximal end to a distal end of the catheter, the catheter comprising a deployable balloon arranged at the distal end of the catheter;
advancing the distal end of the catheter through the opening and into the left atrial appendage;
inflating the balloon to contact an area surrounding the opening of the left atrial appendage to create a seal between the balloon and the left atrial appendage;
evacuating the left atrial appendage to at least partially collapse the left atrial appendage; and
inserting adhesive into the at least partially collapsed left atrial appendage to secure the left atrial appendage in the at least partially collapsed state.

17. The method according to claim 16, wherein the left atrial appendage has a pressure after the balloon contacts an area surrounding the opening of the left atrial appendage, and
wherein evacuating the left atrial appendage comprises creating a reduced pressure in the left atrial appendage to reduce a volume of the left atrial appendage.

18. The method according to claim 16, wherein evacuating the left atrial appendage comprises aspirating through at least one aperture at the distal end of the catheter to create the reduced pressure in the left atrial appendage.

19. The method according to claim 16, wherein the catheter is a cryoballoon catheter, and
wherein inflating the balloon comprises at least partially filling the balloon with cryofluid or refrigerant to contact the area surrounding the opening of the left atrial appendage.

20. The method according to claim 16, the method further comprising incorporating radiopaque markers into the adhesive.

21. The method according to claim 16, the method further comprising curing the adhesive before collapsing the balloon to remove the seal between the balloon and the left atrial appendage.

22. The method of according to claim 16, wherein the adhesive is visible light or ultraviolet cured (UV) adhesive, the method further comprising using a light source to cure the adhesive after the adhesive is inserted into the at least partially collapsed left atrial appendage.

23. The method according to claim 16, wherein the catheter further comprises a securing mechanism arranged to engage with an area surrounding the opening of the left atrial appendage, the method further comprising deploying the securing mechanism to engage with the area surrounding the opening of the left atrial appendage to secure the left atrial appendage in the at least partially collapsed state.

24. The method of according to claim 23, wherein a portion of the distal end of the catheter is detachable and forms the securing mechanism, the method further comprising detaching the portion of the distal end of the catheter and engaging the detached portion with the area surrounding the opening of the left atrial appendage to secure the left atrial appendage in the at least partially collapsed state.
